(19) United States
(12) Patent Application Publication
(10) Pub. No.: US 2023/0167479 A1
(43) Pub. Date: Jun. 1, 2023

(54) RESEARCH INTO ANTIMICROBIAL RESISTANCE BY THE FIELD FLOW FRACTIONATION TECHNIQUE

(71) Applicant: UNIVERSITÉ DE LIMOGES, Limoges Cedex (FR)

(72) Inventors: Serge BATTU, Saint-Victurnien (FR); Gaëlle BÉGAUD, Panazol (FR); Sylvie DELEBASSÉE, Aixe-sur-Vienne (FR); Olivier BARRAUD, Limoges (FR); Raphaël DUVAL, Bioncourt (FR); Philippe CARDOT, Limoges (FR)

(21) Appl. No.: 17/917,231

(22) PCT Filed: Apr. 6, 2021
(86) PCT No.: PCT/IB2021/052832

§ 371 Date: Oct. 5, 2022

(30) Foreign Application Priority Data
Apr. 6, 2020 (FR) 2003429

(51) Int. Cl. C12Q 1/18

(52) U.S. Cl. CPC C12Q 1/18

(57) ABSTRACT

Disclosed is a process for determining the resistance of a microorganism to at least one antimicrobial using a field-flow fractionation device. Also disclosed is the use of a field-flow fractionation device according to the process and a kit for determining the resistance of a microorganism to at least one antimicrobial, using a field-flow fractionation technique.

### CROSS-REFERENCE TO PRIOR APPLICATIONS

This application is the U.S. national phase of International Application No. PCT/IB2021/052832 filed Apr. 6, 2021 which designated the U.S. and claims priority to FR Patent Application No. 2003429 filed Apr. 6, 2020, the entire contents of each of which are hereby incorporated by reference.

### BACKGROUND OF THE INVENTION

### Field of the Invention

The invention relates to the field of biological diagnosis, in particular biomedical diagnosis as part of the research for antimicrobial resistance.

### Description of the Related Art

Various methods are currently implemented for determining the level of sensitivity or resistance of microorganisms which are isolated and identified from samples of different natures (urine, stool, sputum, pus, cerebrospinal fluid, blood, etc.). The reference method is the dilution in a liquid medium, but there are other methods which are automated to a greater or lesser extent and are used in medical biology laboratories, such as microdilution in a liquid medium, diffusion in an agar medium (disk diffusion method) or the “E-tests” technique. For the last two cited methods, the time constraints related to the bacteria growth rates mean that the result of an antibiogram carried out on a given day can be obtained only the next day after 16 to 24 hours of incubation. For the antibiogram by microdilution in a liquid medium, it still takes about ten hours on average before obtaining the result.

Such methods for determining antimicrobial sensitivity/resistance profiles are essential diagnostic tools for the management of patients in human or veterinary medicine. Indeed, the prescription of targeted molecules enables an effective treatment of the infection while limiting the emergence of resistant strains linked to the misuse of antimicrobials and in particular of antibiotics. Excessive and unnecessary prescriptions, prescriptions of insufficiently effective molecules, poor patient compliance and self-medication all contribute to the emergence of resistant strains.

It is becoming primordial in the current context of the increase in antibiotic resistance, of the emergence of new resistances, combined with a very small number of new antimicrobials being marketed and with the lack of therapeutic alternatives (antimicrobial peptides, antibodies, phages, phytotherapy, quorum quenching, nanoparticles, etc.), to propose a new sensitive, rapid and effective method for determining antimicrobial resistance in microorganisms.

The current worldwide problem mainly relates to Gram-negative bacteria such as enterobacteria, Pseudomonas aeruginosa or Acinetobacter baumannii, although issues remain with regard to certain Gram-positive bacteria such as Staphylococcus, Enterococcus, etc.

Currently, the methods involved in the production of antibiograms provide a reliable response, on average 36-48 hours post-sampling. Such delay requires the clinician to choose transiently a so-called probabilistic antibiotic therapy, generally using a broad-spectrum antibiotic, according to preestablished protocols. Such delay is a crucial element in the context of acute pathologies involving hospitalization, and can be life-threatening. The development of new methods for carrying out antibiograms aimed at reducing the existing 12-24-hour delay between the isolation/identification of the bacterium and the result of the antibiogram test becomes a major public health issue.

From an economic point of view, with the current techniques, many consumables are used: cards with freeze-dried antibiotics, antibiotic discs, Petri dishes, and involve a large amount of plastic and a complex management of reagents (expiration date of antibiotics, Petri dishes, waste management, reagent-related vigilance, etc.). The invention proposed herein uses only a few consumables which can be delivered in the form of a kit.

It is thus in a context of optimizing the efficiency and precocity of antibiotic therapy that the research for methods reducing the analysis time of the pathogenic strain becomes essential. Through the joint development of prototypes and sedimentation field-flow fractionation (SdFFF) methodologies suitable for bacterial cell sorting, and through the establishment of robust proof of concept, it is now possible for the present inventors to propose a new antibiogram process which can reduce to a maximum of 24 hours, the rendering of the result to the clinician after the patient was taken in care. Such reduction of the time for obtaining the antibiogram result makes it possible to consider a better therapeutic efficiency, an optimized management of emergencies as well as a reduction in the cost of patient care.

The field of application of such process is very wide and relates to both medical and veterinary diagnosis, and is intended for all public and private biological analysis laboratories. Thus, the present invention, beyond the usefulness thereof within the framework of biomedical diagnostics, can be used in industry, in research laboratories or in certifying agencies during the research and development phases of new therapeutic solutions, by demonstrating the antibacterial potentials thereof.

### SUMMARY OF THE INVENTION

The present inventors propose to use the field-flow Fractionation (FFF) technique developed at the end of 1960s by J. C. Giddings. This method is often presented as one of the most versatile separation methods. Indeed, the wide variety of fields (hydrodynamics, gravity, electric, magnetic, etc.) which can be used, of instrumental configurations and elution modes, make it possible to contemplate an infinite number of experimental conditions to be used for the sorting, the separation and the characterization of polymers, powders, emulsions, colloids, nanoparticles or bioparticles: macromolecules, viruses, organelles and cells ranging in size from 10 nm to 100 µm.

One of the methods, the SdFFF, uses a multigravitational field and remains one of the most widely used FFF methods. This method has high sensitivity in terms of change in size, density, shape or deformability of the analyzed objects, and can be used for an early recording of metabolic changes in the cell population without any specific preparation of the analyzed sample. It is thus possible, by simple comparison of elution profiles or fractograms, to reveal a metabolic modification between an untreated control analytical sample and an analytical sample subject to a biological event. Such tracking or monitoring function has proved to be interesting in the field of oncology by demonstrating in an early and sensitive way, the induction of major phenomena such as apoptosis, differentiation, autophagy, hypoxia, the efficiency of transformation by gene expression modulation vector, etc., suggesting the potential thereof as a pharmacological screening tool.

Thus, the inventors carried out the research work which led to the first demonstration of the subject matter of the invention in the field of diagnosis in microbiology.

### DETAILED DESCRIPTION OF THE INVENTION

The subject matter of the present invention is thus a process for determining the resistance of a microorganism to at least one antimicrobial, characterized in that the process comprises the steps consisting of:
providing at least one microbial population of microorganisms from a biological sample;
treating a portion of each microbial population with the antimicrobial, the other portion being untreated with the antimicrobial;
incubating said one or microbial populations either with or without the antimicrobial, thereby obtaining the treated analytical sample(s) and the control analytical sample(s), respectively;
eluting the analytical sample(s) from the previous step in a field-flow fractionation device;
obtaining the elution profiles of the treated analytical sample(s) and the control analytical sample(s) for each microbial population;
and quantifying the variation of the signals contained in the elution profiles of the control analytical sample(s) and the analytical sample(s) treated with the antimicrobial, and comparing same to a significance threshold;
wherein, when the variation of the signals contained in the one or more elution profiles of the analytical sample(s) treated with the antimicrobial compared with the elution profile of the control analytical sample(s) is greater than the significance threshold, then the microorganism of said microbial population is considered to be sensitive to the antimicrobial.

Furthermore, the constituent microorganism of the microbial population may be any one selected from bacteria, fungi, yeasts, protozoa.

Given its phenotypic character, the invention is not limited to a given resistance mechanism nor to a given microorganism species. The invention does not require a heavy inoculum but an inoculum comparable to the inoculum used by the conventional methods in bacteriology.

Also, the antimicrobial may be any one selected from antibiotics, antifungals, antiparasitic agents.

In a particular embodiment, the microorganism is a bacterium.

The term “bacterium” refers to any type of bacterium, either of the Gram-negative type or of the Gram-positive type.

In such embodiment, the antimicrobial is an antibiotic.

The term “antibiotic” refers to an either natural or synthetic organic substance which exerts its action on the bacteria by destroying same (bactericidal effect) or by inhibiting the growth and multiplication thereof (bacteriostatic effect).

According to one aspect of the invention, the microbial population may come from a biological sample.

The biological sample may further be of human or animal origin; it may also be of environmental origin.

In particular, the biological sample may be a sample of urine, stool, sputum, pus, cerebrospinal fluid, of blood or the like.

According to another aspect of the invention, the biological sample may be a reference strain.

The term “reference strain” refers to an isolated microorganism strain the characters of which have been studied and which is preserved over a long period of time in a library of reference strains.

The two aspects, namely a biological sample and a reference strain, constitute the “biological samples” as defined by the present invention.

Such biological samples are grown in a culture in agar medium in order to obtain a “microbial population”, whether a bacterial, parasitic, fungus, yeast, etc. population.

The microbial population as defined by the present invention may thus be a microbial suspension obtained from a mixture of visually identical colonies isolated from a biological sample.

Such suspension, considered visually as homogeneous, is then incubated in the presence of antimicrobials of various natures at different concentrations (treated) or incubated for the same duration in the absence of any antimicrobial (control). These samples are the “analytical samples” which will be eluted in the field-flow fractionation device.

According to a particular embodiment, the incubation step with the antimicrobial may last from 30 to 120 minutes, preferably from 30 to 60 minutes.

Moreover, the incubation step with the antimicrobial is carried out in a liquid culture medium.

The field-flow fractionation device used in the method of the invention may further be a multigravitational or centrifugal, hydrodynamic, dielectrophoretic (DEP), electric, magnetic, thermal field-flow fractionation device, or any other type of suitable field-flow fractionation device.

“Multigravitational”, “sedimentation” or “centrifugal” FFF (SdFFF or CFFF) refers to field-flow fractionation where the applied field is gravitational with a force > 1 g. Such field is obtained by rotating the separation channel. The field strength is proportional to the rotation speed of the separation channel.

“Hydrodynamic” FFF (FlFFF) refers to field-flow fractionation where the field is hydrodynamic, either in a symmetrical or asymmetrical flow channel (Asymmetrical FFF) and where only the accumulation wall consists of a semi-permeable membrane.

“Electric” FFF (ElFFF) refers to field-flow fractionation where the applied field is electric, continuous or cyclic. To this end, the walls of the separation channel are electrodes.

“Magnetic” FFF (MgFFF) refers to field-flow fractionation where the applied field is magnetic.

“Thermal” FFF (ThFFF) refers to field-flow fractionation where the field is thermal, corresponding to a temperature gradient between the two walls of the separation channel.

“Dielectrophoretic” FFF (DEP-FFF) refers to field-flow fractionation where the field is a dielectrophoresis field.

In a particular embodiment of the present invention, the field-flow fractionation device is a multigravitational field-flow fractionation device.

According to this aspect, the field-flow fractionation device involves a step of stopping the mobile phase flow, commonly called stop-flow.

The “stop-flow” step corresponds to the step of primary concentration of the sample in the separation channel. Following the injection of the sample, the mobile phase flow conducts the species to be separated into the channel. Knowing the separation system (volume of the injection loop, volume of the tubes) and the flow rate applied, it is possible to calculate the time required for the species to enter the channel. When the species are in position within the channel, the mobile phase flow is cut off, and only the multigravitational force is applied to the species, which are led to the accumulation wall where same concentrate, that is the primary focusing. After a sufficient time of absence of flow (=stop-flow), the mobile phase flow is applied again, the ascending hydrodynamic forces rise again, thus leading the species to their equilibrium position for their separation, namely the secondary focusing.

“Elution profile” or “Fractogram” refers to the diagram which represents the instantaneous variation in concentration or quantity of the species at the outlet of the separation channel as a function of the progress of the analysis = (concentration/quantity of the eluted species) = f (elution time or volume). In most cases, the fractogram consists of two major peaks, the first corresponding to the dead volume (species not retained, elution time = t₀), the second corresponding to the peak of the microbial population (species retained, elution time = tᵣₙ > t₀).

According to the present invention, the non-retained species correspond to molecules coming from the culture medium, to biological or cellular debris, etc., which absorb the UV signal of the detector, but which are not sensitive to the field applied. For example, in the case of SdFFF, at the rotation speeds which are applied, and thus at the gravitational fields used (10-50 g), molecules with a size smaller than the µm, do not undergo the effect of the field: said molecules are thus not retained. Said molecules advance through the system at the same speed as the mobile phase, i.e. the liquid carrier within the device. If the time required for the elution of such species is measured = dead time or t₀, it is the time the mobile phase needs for traveling through the field-flow fractionation device from the injection system (Rheodyne® valve) to the detector (UV-Vis spectrophotometer) by flowing through the tubes and the separation channel inserted into the centrifuge bowl.

Furthermore, the signal(s) contained in the elution profile(s) of the analytical sample(s) which are analyzed with regard to the variability thereof are namely the peak position defined by the tᵣ measured at the peak apex, or defined by the peak median, or normalized by the calculation of the retention factor, R_{obs} = tᵣₙ/t₀, or any other method of determination, or the peak width of the elution profile.

The variation of the signals contained in the elution profiles will then be quantified. It is expressed in % and corresponds to a percentage of variation in Robs = PΔR; signifying a biological effect, i.e. the percentage of variation in the retention factor

$$\text{P} \text{Δ} \text{R} = \frac{\left|\text{Δ} {\text{R}}_{\text{Obs}}\right|}{{\text{R}}_{{\text{Obs}}_{\text{control}}}} \times 100$$

The “significance threshold” is the threshold from which the microbial population is considered to be sensitive or resistant to the antimicrobial. The value thereof is defined for every microorganism/antimicrobial pair, as a function of the usual active doses of the antimicrobial. The values will be referenced in a dedicated database.

If the measured value of PΔR is < to the significance threshold, then the microorganism is considered to be resistant. If the measured value of PΔR is > than the significance threshold, then the microorganism is considered to be sensitive. The degree of sensitivity of the detection method makes possible the identification of profiles which are “sensitive” and “resistant” to an antimicrobial.

By way of example, PΔR has the following values for reference strains of E. coli:

E.g. Ampicillin:
sensitive strain incubated with 4 mg/ml ≈ 13%
Resistant strain incubated with 4 mg/ml ≈ 5%
Resistant strain incubated with 8 mg/ml ≈ 7%

For this strain, the analyzes allowed to define the significance threshold as being > 10%

According to the invention, the significance threshold is defined for each microorganism/antimicrobial pair.

The invention also relates to the use of the field-flow fractionation device according to the process for determining the resistance of a microorganism of the present invention.

The invention also relates to a kit for determining the resistance of a microorganism to at least one antimicrobial by a field-flow fractionation technique comprising:
at least one antimicrobial selected from antibiotics, antifungal agents, antiparasitic agents;
optionally at least one tube for the incubation of the microbial population;
optionally at least one component selected from a separation channel, rotary joints, tubing, semipermeable membranes or other components of the field-flow fractionation device;
optionally one or more solutions for cleaning and decontaminating said channels and/or rotary joints and/or tubing;
optionally instructions for the use of the kit.

According to such embodiment, the components of the kit consisting in particular of the separation channel, the rotary joints, the tubing and the semipermeable membranes are provided for single-use.

However, it is intended to provide in the kit, one or more solutions for cleaning and decontaminating the components in order to reduce the ecological impact and render same reusable. It is contemplated that at least two series of rolling kits can be used, one in use and the other being cleaned and decontaminated. Also, the used kits can be recovered by the manufacturer so as to ensure the recycling and reconditioning thereof, always in an optic to reduce the ecological and economic impact.

“Separation channel” refers to the flow channel on which an external field of variable nature is applied depending on the type of FFF used and wherein circulate the analyzed microorganisms.

It may be made of a sheet of plastic material, most generally mylar, wherein the shape of the channel is cut. In general, the channel has an either a trapezoidal or a parallelepipedal shape with V-shaped tips. The length (conventionally, 15 to 80 cm) and the width (conventionally, 8 to 20 mm) of the shape cut from the sheet of plastic material defines the length and the width of the channel. The thickness of the mylar sheet defines the thickness of the channel (conventionally, 125 to 350 µm). The sheet is then placed between two walls so as to define the volume of the channel, to provide leak-tightness and the passing of the mobile phase and of the samples.

The walls of the separation channel vary depending on the type of FFF. In the case of SdFFF, the walls are non-permeable and rigid. Such type of walls is shared with MgFFF. For ThFFF, the walls are non-permeable plates which are heated to different temperatures. For ElFFF, the walls are electrodes. For FlFFF, at least one of the two walls is a wall associated with a semi-permeable membrane and consists of sintered glass.

“Rotary joints” refers to the device for the passing of the mobile phase and of the samples at the inlet and outlet of the separation channel from a fixed reference frame (namely a mobile phase pump, sample injector, sample detector) to a rotating reference frame (the separation channel). The passing has to take place without any leakage of liquid and hence risk of dispersion of the sample, and the potential contamination of the operator. Rotary joints are strategic parts in an SdFFF device.

“Tubing” refers to the tubes leading the samples from the sample injector to the separation channel as well as to the tubes bringing the separated species from the separation channel to the detector.

The detector differs depending on the type of FFF technique. The most commonly used detector is a UV-Vis spectrophotometer detector used for the measurement of the absorbance variation over time.

Thus, the present invention relates in particular to a kit for determining the resistance of a bacterial population to an antibiotic using an SdFFF fractionation technique comprising:
at least one antibiotic;
optionally, at least one tube for the incubation of the biological population;
at least one separation channel;
optionally, at least one component selected from rotary joints and tubing of the gravitational field-flow fractionation device;
optionally, one or more solutions for cleaning and decontaminating said channels and/or rotary joints and/or tubing;
optionally, instructions for the use of the kit.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to better illustrate the subject-matter of the present invention, a particular embodiment will be described hereinafter, as an indication, but not limited to, with reference to the enclosed figures.

In these figures:

FIG. 1 shows the analytical diagram for measuring resistance to an antibiotic according to the implementation of Example 1.

FIG. 2 shows the fractogram obtained during the test of the sensitivity of the E. coli strain ATCC 25922 to kanamycin (0/1.5/3/6/12 µg/ml) according to Example 1.

FIG. 3A shows the fractograms obtained during the test of the sensitivity of the E. coli strain ATCC 25922 to chlortetracycline according to Example 1.

FIG. 3B shows the fractograms obtained during the test of the sensitivity of the E. coli strain ATCC 25922 to the cinnamon essential oils according to Example 1.

FIG. 3C shows the fractograms obtained during the test of the sensitivity of the E. coli strain ATCC 25922 to oregano essential oils according to Example 1.

FIG. 4 shows the analytical diagram for measuring resistance to an antibiotic according to the implementation of Example 2.

FIG. 5 shows the fractograms obtained during the test of the sensitivity to ampicillin of the E. coli strain ATCC 35218 (A) with regard to the E. coli strain ATCC 25922 (B) demonstrating the resistance of the strain 35218 unlike the strain 25922, according to Example 2.

FIG. 6 shows the analytical diagram of the process for detecting the resistance to an antibiotic according to the present invention (A) compared to the current techniques (B).

### DESCRIPTION OF THE PREFERRED EMBODIMENTS

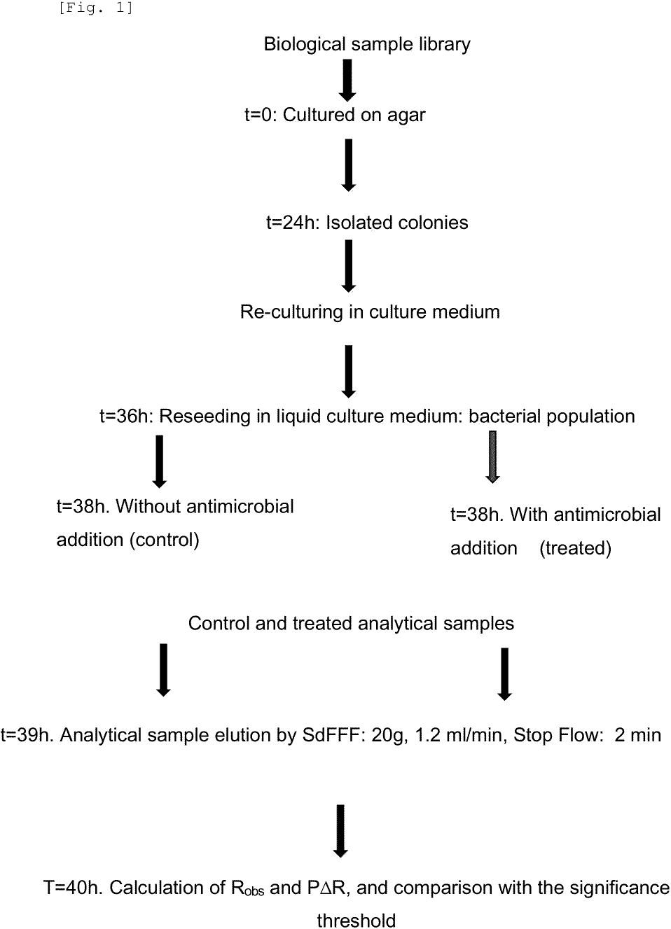

FIG. 1 shows the summary of the protocol of the detection method according to the implementation of Example 1. The results of the antimicrobial resistance analysis are expected starting 40 hours after the sample was cultured on agar.

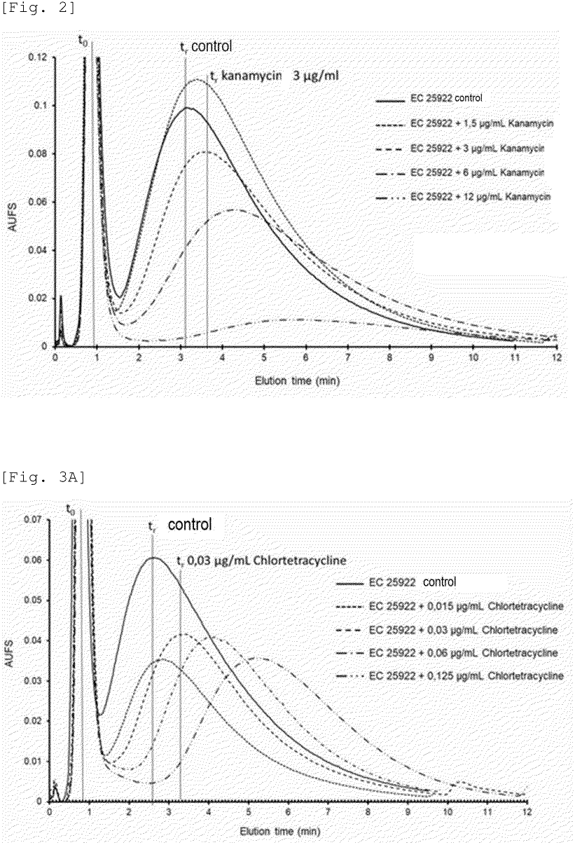

FIG. 2 shows the overlay of 5 elution profiles obtained for the reference strain E. coli ATCC EC 25922, incubated in the absence (control) or in the presence of increasing doses of kanamycin (1.5 to 12 µg/ml) according to the protocol described in FIG. 1. The elution profiles or fractograms were produced under the elution conditions also described in FIG. 1. The value of t₀ is on average 0.96 minute. By way of example, the retention times (tᵣ) are indicated for the control conditions (on average 3.22 minutes) and treated with 3 µg/ml of kanamycin (3.62 minutes on average) making possible the calculations of R_{obs} for the control (0.299) and the treated samples (0.265 for 3 µg/ml), which corresponds to a percentage of variation in the retention factor PΔR = 11.2%; 10% can be considered to be the significance threshold. At such concentration, which corresponds to the minimum inhibitory concentration of kanamycin, the strain is well sensitive to the antimicrobial.

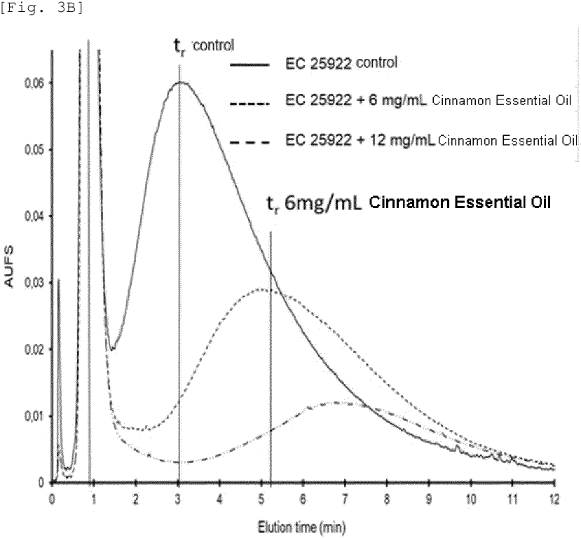

Similarly, FIG. 3A shows the overlay of 5 elution profiles obtained for the reference strain E. coli ATCC EC 25922, incubated in the absence (control) or in the presence of increasing doses of chlortetracycline (0.015 to 0.125 µg/ml) according to the protocol described in FIG. 1. The elution profiles or fractograms were produced under the elution conditions also described in FIG. 1. The value of t₀ is on average 0.96 minute. By way of example, the retention times (tᵣ) are indicated for the control conditions (on average 2.62 minutes) and treated with 0.03 pg/ml of chlortetracycline (3.36 minutes on average) making possible the calculations of R_{obs} for the control (0.367) and the treated samples (0.286 for 0.03 µg/ml), which corresponds to a PΔR = 22%; 10% can be considered to be the significance threshold. At such concentration, which corresponds to the minimum inhibitory concentration of chlortetracycline, the strain is well sensitive to the antimicrobial.

Similarly, FIGS. 3B and 3C show that the E. coli 25922 strain is also significantly sensitive to the action of cinnamon essential oil since PΔR=40%, to the action of oregano essential oil since PΔR=13.9%.

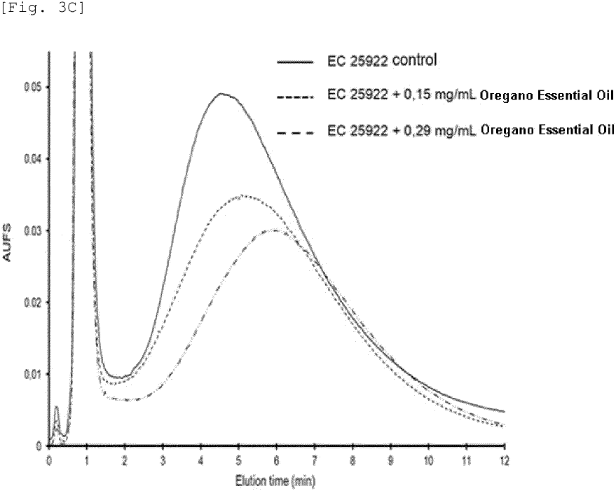

FIG. 4 shows the summary of the protocol of the detection method according to the implementation of Example 2. The results of the antimicrobial resistance analysis are expected starting 18-26 hours after the sample was cultured on agar.

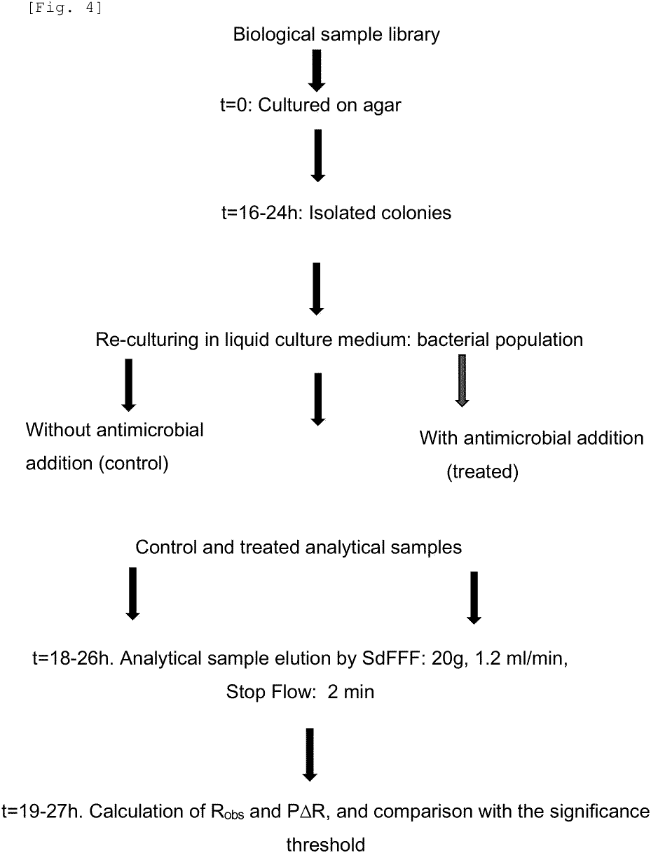

FIG. 5A shows 3 elution profiles obtained for the reference strain E. coli ATCC EC 35218, incubated in the absence (control) or in the presence of increasing doses of ampicillin (4 or 8 mg/ml) according to the protocol described in FIG. 4. The elution profiles or fractograms were produced under the elution conditions also described in FIG. 4. The value of t₀ is on average 0.96 minute. By way of example, the retention times (tᵣ) are indicated for the control conditions (on average 3.74 minutes) and treated with 4 mg/ml of ampicillin (3.58 minutes on average) making possible the calculations of R_{obs} for the control (0.257) and the treated samples (0.269 for 4 mg/ml), which corresponds to a percentage of variation in the retention factor PΔR = 4.50; 10% can be considered to be the significance threshold.

At such concentration, the strain is resistant to the antimicrobial.

Similarly, FIG. 5B shows the elution profiles of the reference strain E. coli ATCC EC 25922, incubated in the absence (control) or in the presence of increasing doses of ampicillin (4 or 8 mg/ml). It can be seen that, unlike the strain E. coli ATCC EC 35218, E. coli 25922 is sensitive to ampicillin. Indeed, the retention times (tᵣ) for the control conditions (on average 2.66 minutes) and treated with 4 mg/ml of ampicillin (2.36 minutes on average) making possible the calculations of R_{obs} for the control (0.362) and the treated samples (0.408 for 4 mg/ml), which corresponds to a percentage of variation in the retention factor PΔR = 12.70; 10% can be considered to be the significance threshold. At such concentration, the strain is sensitive to the antimicrobial.

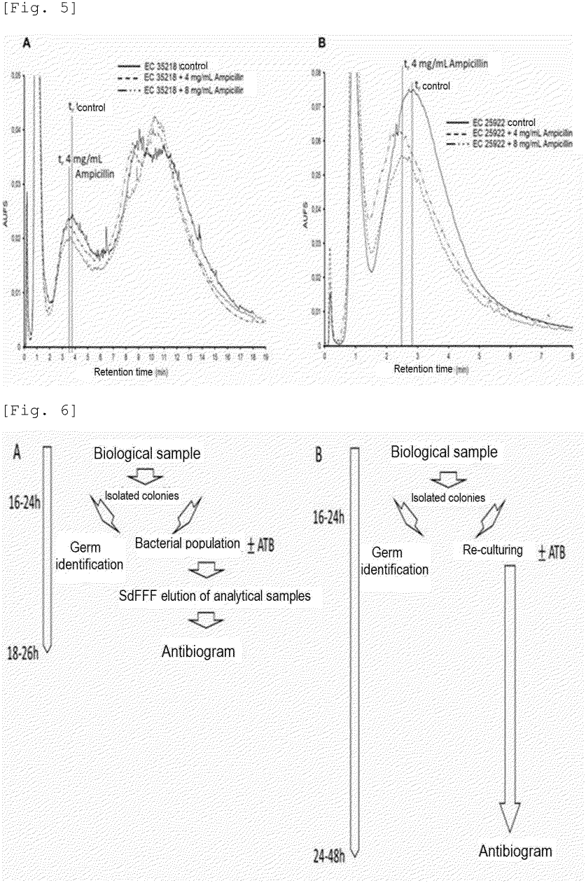

Finally, FIG. 6 shows the time saving made possible by the present invention compared with current techniques. Such significant time saving is a major advance in detection techniques for antimicrobial resistance in microorganisms. This makes it possible for a clinician or a veterinarian to be directed early on toward the most appropriate therapeutic approach.

### EXAMPLES

### Materials & Methods

The field-flow fractionation device is set according to patent EP1679124. The device comprises an injection system where the sample is taken, the tubing bringing the sample from the injector to the separation channel via a first rotary joint, the separation channel contained in a centrifugation bowl, the rotation of which is ensured by an electric motor itself controlled manually or via a suitable computer device, thus allowing control of the intensity of the multigravitational field, the tubing bringing the separated species from the channel to the detector via the second rotary joint, and finally the detector.

Culture medium: DIFCO™ Mueller Hinton Broth (Becton Dickinson, reference 275730)

Bacterial strains used: E. coli 25922 and 35218 (ATCC, Manassas, VA, USA).

Antibiotics: Ampicillin (Sigma-Aldrich, Saint-Quentin-Fallavier, France, ref. 21442020), kanamycin (MP Biomedical, Illkirch FRANCE, ref. 150020) Chlortetracycline (Sigma-Aldrich, Saint-Quentin-Fallavier, France, Ref 17776).

### Culture Conditions

Bacterial colonies were incubated in a Mueller-Hinton (MH) culture medium with an inoculum of 0.5 McFarland in the absence or in the presence of antibiotic, for 2 hours at 37° C. and under stirring. The liquid medium MH is the reference medium used for the reference method for determining MIC values, called the dilution method in a liquid medium.

After incubation for 2 h, the culture medium was centrifuged and then taken up in a volume of 0.5 ml of PBS.

Example of calculation of the percentage of variation of Robs:

According to the handlings shown in FIG. 5, the calculation of the percentage of variation in the retention factor or PΔR is carried out as follows:

[symbol]

$$P Δ R = \frac{\left|{\text{R}}_{{\text{Obs}}_{\text{control}}} - {\text{R}}_{{\text{Obs}}_{\text{treated}}}\right|}{{\text{R}}_{{\text{Obs}}_{\text{control}}}} \times 100 = \frac{\left|\text{Δ} {\text{R}}_{\text{Obs}}\right|}{{\text{R}}_{{\text{Obs}}_{\text{control}}}} \times 100 ;$$

Table 1: Results of the analysis of fractograms according to the experiment illustrated in FIGS. 5A and 5B: calculation of the PΔR for determining, by comparison with the significance threshold, set at 10% for such strains and such antibiotic, the resistance of the strains of E. coli 25922 and 35218 to Ampicillin

Example 1: Results of resistance tests of the E. coli strain ATCC 25922 to various molecules, using SdFFF according to the present invention.

These results made it possible to establish a first protocol for the preparation of the population of microorganisms, in the present case, the bacterial suspension (FIG. 1).

The SdFFF elution conditions were adapted from the conditions generally used in cell sorting practices for eukaryotic cells. The direct introduction of the sample in the opposite direction to the gravity field, enabling a rapid relaxation of the species in the flow lines effective for the separation thereof, is herein optimized by a brief additional stop-flow step, due to the small sizes (< 2 µm) of the species to be separated.

The basic conditions were established as follows:
Mobile phase flow rate: 1.2 ml/minute;
Multi-gravitational field: 20 g;
Stop-flow: 2 minutes.

With such conditions, it was possible to obtain a response after only 1 hour of incubation.

The results (FIGS. 2 and 3A) on the E. coli ATCC 25922 strain model with respect to kanamycin (increasing concentration from 0 to 12 µg/ml) and chlorotetracycline (0.015 to 0.125 µg/ml) make it possible to demonstrate the ability of the SdFFF apparatus to record retention changes, namely responses which intensity varies depending on the concentration of the antibiotic used.

These results show that the detection process of the present invention enables a quantitative, dose-dependent response which is complementary to its qualitative dimension, namely the measurement of the significance threshold of resistance vs. sensitivity to the antimicrobial. The degree of sensitivity of the detection process is based on the ability of the FFF device to record retention variations depending on the concentration of antimicrobial used.

In said figures, a decrease in the retention factor = R_{obs} = t₀ /trₙ is observed with regard to the elution peak of the bacteria, which is characteristic of the induction of the biological event linked to the incubation in the presence of antibacterial agents. Such very encouraging results made it possible to consider reducing by about 24 hours the time required to obtain the result of an antibiogram compared to the existing methods.

Example 2: A new analysis protocol was established according to FIG. 4, consistent with the clinical implementation of conventional antibiograms, making it possible to reduce to 26 hours post-sampling/isolating the time for obtaining the test results.

Conventionally, an antibiogram is carried out from bacterial colonies obtained from a biological sampling. The time to obtain such colonies is on average 16-24 hours, but it can sometimes be shorter, on the range of 10-12 hours depending on the bacterial species. Such colonies are then placed back in suspension in liquid medium and incubated for 16-24 hours with different antibiotics according to the recommendations in force of the CA-SFM (Antibiogram Committee of the French Society of Microbiology).

Thus, such assays were carried out directly on bacterial suspensions, obtained from colonies of the E. coli strain ATCC 25922 and ATCC 35218 cultured for 16-24 hours on agar medium, and incubated for 2 hours in the presence or in the absence of antibiotic (Ampicillin, FIGS. 5A/B).

It was thus possible to confirm the dose-dependent variation of the factor R_{obs} for an antibiotic, on different strains (sensitive/resistant) of E. coli bacteria.

The difference in behavior of the bacterial strain is demonstrated by the measurement of R_{obs}, the calculation of PΔR and the comparison thereof with significance thresholds making it possible to indicate the sensitivity or the resistance of the bacterial strain to the bactericidal or bacteriostatic action of the antibiotic tested.

Antimicrobial resistance is measured by comparing PΔR with the significance threshold, PΔR being expressed according to the formula below: Considering the

$$\text{P} \text{Δ} \text{R} = \frac{\left|\text{Δ} {\text{R}}_{\text{Obs}}\right|}{{\text{R}}_{{\text{Obs}}_{\text{control}}}} \times 100 ,$$

if the measured value PΔR < significance threshold; then the microorganism is considered to be resistant.

Similarly, if the measured value PΔR > significance threshold; then the microorganism is considered to be sensitive.

Such measurement, based only on variations in intrinsic biophysical properties related to the action of the antibiotic on the bacterial strain, does not involve any specific sample preparation, nor any specific or expensive reagent. Taking into account the sensitivity of the separation method to variations in such parameters (size, density, shape, deformability, motility, etc.), the reading of the antibiogram can be done very early (24 hours after recovery of the pathological sampling and isolation and identification of the germ), making it possible to provide a diagnosis to the clinician with a gain of nearly 24 hours over the current protocols.

FIG. 6 shows the general analytical diagram of the process according to the invention and display same in parallel with the protocols currently available in the prior art.

The inventive character of the present invention lies in the simplicity and speed of its implementation. The process has the advantage of enabling the automation of the steps, from the automatic injection of the samples into the device until the result is obtained in terms of sensitivity/resistance. The apparatus is intended for being specially designed for facilitating maintenance, sterile and safe handling for the operator, and a rapid change of the separation channel, a part designed for single-use, e.g. in a kit according to the invention: one channel = one microorganism = one antibiogram.

The simplicity and speed of the process according to the invention are based on the fact that the process is completely devoid of any prior treatment of the sample to be eluted, leading to the results being obtained more quickly.

The process according to the invention also has an aspect of universality and is applicable to any type of microorganism, namely bacteria, fungi, yeasts, protozoa. It is not previously required to identify a specific antigen for separating the microbial populations as is required with flow cytometry.

The field-flow fractionation technique has no a priori and is applicable regardless of the nature of the microorganism.

It is understood that the above embodiments of the present invention have been given as an indication, and are non-limiting, and that modifications can be made thereto without departing from the scope of the present invention. In particular, other measurement ranges, precisions, dimensions and features of the means of data acquisition and conditions can be selected depending on the intended use.

1. A process for determining the resistance of a microorganism to at least one antimicrobial, wherein the process comprises the steps consisting of:
providing at least one microbial population of microorganisms from a biological sample;
treating a portion of each microbial population with the antimicrobial, the other portion being untreated with the antimicrobial;
incubating the at least one microbial populations either with or without the antimicrobial, thereby obtaining the treated analytical sample(s) and the control analytical sample(s), respectively;
eluting the analytical sample(s) from the previous step in a field-flow fractionation device;
obtaining the elution profiles of the treated analytical sample(s) and the control analytical sample(s) for each microbial population;
and quantifying the variation of the signals contained in the elution profiles of the control analytical sample(s) and the analytical sample(s) treated with the antimicrobial, and comparing the variation of the signals to a significance threshold;
wherein when the variation of the signals contained in the at least one elution profiles of the analytical sample(s) treated with an antimicrobial compared to the elution profile of the control analytical sample(s) is greater than the significance threshold, then the microorganism of the microbial population is considered to be sensitive to the antimicrobial.

2. The process for determining the resistance of a microorganism to at least one antimicrobial according to claim 1, wherein the microorganism is one selected from bacteria, fungi, yeasts, protozoa.

3. The process for determining the resistance of a microorganism to at least one antimicrobial according to claim 1, wherein the antimicrobial is one selected from antibiotics, antifungals, antiparasitic agents.

4. The process for determining the resistance of a microorganism to at least one antimicrobial according to claim 1, wherein the microorganism is a bacterium.

5. The process for determining the resistance of a microorganism to at least one antimicrobial according to claim 4, wherein the antimicrobial is an antibiotic.

6. The process for determining the resistance of a microorganism to at least one antimicrobial according to claim 1, wherein the microbial population comes from a biological sample.

7. The process for determining the resistance of a microorganism to at least one antimicrobial according to claim 6, wherein the origin of the biological sample is one of human, animal and environmental origin.

8. The process for determining the resistance of a microorganism to at least one antimicrobial according to claim 6, wherein the biological sample is a sample of urine, stool, sputum, broncho-alveolar washing, pus, cerebrospinal fluid, or blood .

9. The process for determining the resistance of a microorganism to at least one antimicrobial according to claim 1, wherein the biological sample is a reference strain.

10. The process for determining the resistance of a microorganism to at least one antimicrobial according to claim 1, wherein the incubation step with the antimicrobial lasts from 30 to 120 minutes.

11. The process for determining the resistance of a microorganism to at least one antimicrobial according to claim 1, wherein the field-flow fractionation device is a multigravitational or centrifugal, hydrodynamic, dielectrophoretic (DEP), electric, magnetic, thermal field-flow fractionation device .

12. The process for determining the resistance of a microorganism to at least one antimicrobial according to claim 1, wherein the field-flow fractionation device is a multigravitational field-flow fractionation device.

13. The process for determining the resistance of a microorganism to at least one antimicrobial according to claim 1, wherein the signal or signals contained in the elution profile(s) of the analytical sample(s) which are analyzed with regard to the variability thereof, are in particular the peak position defined by the tᵣ measured at the peak apex, or defined by the peak median, or normalized by the calculation of the retention factor, R_{obs} = tᵣₙ/t₀ or any other method of determination, or the peak width of the elution profile.

14. The process for determining the resistance of a microorganism to at least one antimicrobial according to claim 1, wherein the significance threshold is defined for each microorganism/antimicrobial pair.

15. (canceled)

16. Kit for determining the resistance of a microorganism to at least one antimicrobial by a field-flow fractionation technique comprising:
at least one antimicrobial selected from antibiotics, antifungal agents, antiparasitic agents;
at least one tube for the incubation of the microbial population;
at least one component selected from a separation channel, rotary joints, tubing, semipermeable membranes or other components of the field-flow fractionation device;
at least one solutions for cleaning and decontaminating the channels and/or rotary joints and/or tubing;
instructions for use of the kit.

17. A kit for the determination of the resistance of a bacterial population to an antibiotic using an SdFFF fractionation technique comprising:
at least one antibiotic;
at least one tube for the incubation of the biological population;
at least one separation channel;
at least one component selected from rotary joints and tubing of the gravitational field-flow fractionation device;
at least one solution for cleaning and decontaminating the channels and/or rotary joints and/or tubing;
instructions for use of the kit.

18. The process for determining the resistance of a microorganism to at least one antimicrobial according to claim 2, wherein the antimicrobial is one selected from antibiotics, antifungals, antiparasitic agents.

19. The process for determining the resistance of a microorganism to at least one antimicrobial according to claim 2, wherein the microorganism is a bacterium.

20. The process for determining the resistance of a microorganism to at least one antimicrobial according to claim 3, wherein the microorganism is a bacterium.

21. The process for determining the resistance of a microorganism to at least one antimicrobial according to claim 2, wherein the microbial population comes from a biological sample.
